(19) United States
(12) Patent Application Publication
(10) Pub. No.: US 2024/0284947 A1
(43) Pub. Date: Aug. 29, 2024

(54) ELECTROSPUN POLYMER FIBERS FOR CULTURED MEAT PRODUCTION

(71) Applicant: NFS IP Holdings, LLC, Dublin, OH (US)

(72) Inventors: Jed K. JOHNSON, London, OH (US); Devan OHST, Columbus, OH (US)

(73) Assignee: NFS IP Holdings, LLC, Dublin, OH (US)

(21) Appl. No.: 18/479,870

(22) Filed: Oct. 3, 2023

Related U.S. Application Data
(63) Continuation of application No. 16/780,187, filed on Feb. 3, 2020, now abandoned.
(60) Provisional application No. 62/800,051, filed on Feb. 1, 2019.

(51) Int. Cl. A23L 13/40; A23L 13/00; C12N 5/00; C12N 5/071

(52) U.S. Cl. CPC A23L 13/45; A23L 13/00; C12N 5/0068; C12N 5/0602; A23V 2002/00; C12N 2533/30; C12N 2533/50

(57) ABSTRACT

A cultured meat product may comprise a scaffold comprising an electrospun polymer fiber, and a population of cells. The cultured meat product may have a thickness from about 100 μm to about 500 mm. A method of producing such a cultured meat product may comprise preparing the scaffold, placing the scaffold into a bioreactor, adding the population of cells to the bioreactor, culturing the population of cells in the bioreactor containing the scaffold for a period of time, thereby forming the cultured meat product, and removing the cultured meat product from the bioreactor. The cultured meat product may be configured to mimic the taste, texture, size, shape, and/or topography of a traditional slaughtered meat.

### CROSS-REFERENCE TO RELATED APPLICATIONS

This application is a continuation of U.S. patent application Ser. No. 16/780,187, filed Feb. 3, 2020, which claims priority to U.S. Provisional Application No. 62/800,051, filed Feb. 1, 2019, both of which are entitled “Electrospun Polymer Fibers for Cultured Meat Production,” and both of which are incorporated herein by reference in their entirety.

### BACKGROUND

The concept of lab-grown meat originally arose from space travel research. It was suggested that if meat could be grown in vitro, astronauts could grow their food to sustain longer space voyages. The idea was simple: culture mesenchymal stem cells into muscle, fat, and connective tissue to create an alternative to slaughtered meat. Since the concept was initially explored, several entities have begun researching and developing ways to commercialize cultured, or “clean,” meats. Motivations for this research include ideas of sustainability, animal welfare, carbon emissions, and consumer health.

Several companies have successfully developed cell biology methods to grow a product that includes muscle, fat, and/or connective tissue, but all of these products are limited to the traditional yields of a petri dish or test tube. When most cells are cultured in a dish, for example, they form only a monolayer, and the surface area of the layer is limited by the size of the dish or the number of cells. The cells in these cultures lack the necessary nutritional environment to properly stack on top of one another, making it implausible to expect a noticeable volume or thickness increase from traditional cell culture techniques. This implausibility drastically affects the quality of and potential for cultured meat products. These cultured cells also generally lack the taste and texture of slaughtered meat. Therefore, there exists a need for the production of a thicker lab-cultured “clean” meat product with improved taste and texture.

### SUMMARY

In an embodiment, a cultured meat product may comprise a scaffold comprising an electrospun polymer fiber, and a population of cells. The cultured meat product may have, in some embodiments, a thickness from about 100 μm to about 500 mm. In an embodiment, a method of producing such a cultured meat product may comprise preparing the scaffold, placing the scaffold into a bioreactor, adding the population of cells to the bioreactor, culturing the population of cells in the bioreactor containing the scaffold for a period of time, thereby forming the cultured meat product, and removing the cultured meat product from the bioreactor. In some embodiments, the cultured meat product may be configured to mimic the taste, texture, size, shape, and/or topography of a traditional slaughtered meat.

### BRIEF DESCRIPTION OF THE DRAWINGS

FIG. 1A shows an SEM image (8900×) of an embodiment of a scaffold as described herein, the scaffold electrospun using a 100k Mw PEO+zein solution.

FIG. 1B shows an SEM image (1700×) of the scaffold of FIG. 1A.

FIG. 2A shows an SEM image (1500×) of an embodiment of a scaffold as described herein, the scaffold electrospun using a 1M Mw PEO+zein solution.

FIG. 2B shows an SEM image (200×) of the scaffold of FIG. 2A.

FIG. 3A shows an SEM image (5000×) of an embodiment of a scaffold as described herein, the scaffold electrospun using a PDLGA 5010+zein solution.

FIG. 3B shows an SEM image (1650×) of the scaffold of FIG. 3A.

FIG. 4A shows an SEM image (2150×) of an embodiment of a scaffold as described herein, the scaffold electrospun using a PCL+soy protein isolate solution.

FIG. 4B shows an SEM image (215×) of the scaffold of FIG. 4A.

### DETAILED DESCRIPTION

This disclosure is not limited to the particular systems, devices, and methods described, as these may vary. The terminology used in the description is for the purpose of describing the particular versions or embodiments only, and is not intended to limit the scope of the disclosure.

The following terms shall have, for the purposes of this application, the respective meanings set forth below. Unless otherwise defined, all technical and scientific terms used herein have the same meanings as commonly understood by one of ordinary skill in the art. Nothing in this disclosure is to be construed as an admission that the embodiments described in this disclosure are not entitled to antedate such disclosure by virtue of prior invention.

As used herein, the singular forms “a,” “an,” and “the” include plural references, unless the context clearly dictates otherwise. Thus, for example, reference to a “fiber” is a reference to one or more fibers and equivalents thereof known to those skilled in the art, and so forth.

As used herein, the term “about” means plus or minus 10% of the numerical value of the number with which it is being used. Therefore, about 50 mm means in the range of 45 mm to 55 mm.

As used herein, the term “consists of” or “consisting of” means that the device or method includes only the elements, steps, or ingredients specifically recited in the particular claimed embodiment or claim.

In embodiments or claims where the term comprising is used as the transition phrase, such embodiments can also be envisioned with replacement of the term “comprising” with the terms “consisting of” or “consisting essentially of.”

As used herein, the term “traditional slaughtered meat” means one or more types of meat obtained from a once-living animal for the purpose of consumption. Such meat is generally, although not always, obtained from livestock, fish, or other animals raised or slaughtered primarily for food production purposes. Non-limiting examples of traditional slaughtered meat include chicken, turkey, pork, steak, fish, and the like. Traditional slaughtered meat is generally appropriate for consumption by one or more mammal species.

As used herein, the term “cultured meat product” means a meat product that is produced by human or machine intervention, rather than grown as a natural component of a living animal. A cultured meat product is thus not obtained directly from the slaughter of a living animal. Like traditional slaughtered meat, a cultured meat product is generally appropriate for consumption by one or more mammal species.

The concept of lab-grown meat originally arose from space travel research. It was suggested that if meat could be grown in vitro, astronauts could grow their food to sustain longer space voyages. The idea was simple: culture mesenchymal stem cells into muscle, fat, and connective tissue to create an alternative to slaughtered meat. Since the concept was initially explored, several entities have begun researching and developing ways to commercialize cultured, or “clean,” meats. Motivations for this research include ideas of sustainability, animal welfare, carbon emissions, and consumer health.

Several companies have successfully developed cell biology methods to grow a product that includes muscle, fat, and/or connective tissue, but all of these products are limited to the traditional yields of a petri dish or test tube. When most cells are cultured in a dish, for example, they form only a monolayer, and the surface area of the layer is limited by the size of the dish or the number of cells. This occurs because cells want to attach to a surface, so they will adhere to the plastic bottom of a dish or flask. Cells migrate in search of more surface area to attach to, and at some point, these cells reach maximum confluence as a monolayer. The cells in these cultures lack the necessary nutritional environment to properly stack on top of one another, although there are some cell lines that can potentially stack to form one or two additional layers in the presence of the correct signaling factors. Even so, it is implausible to expect a noticeable volume or thickness increase from traditional cell culture techniques, and this implausibility drastically affects the quality of and potential for cultured meat products. Companies currently developing these “clean” meat products tend to face similar engineering challenges.

These cultured cells also generally lack the taste and texture of slaughtered meat. This lack of taste and texture follows from the above-mentioned cell culture limitations. Traditional slaughtered meat grows naturally with correct fiber alignments, vascularization, and additional factors that contribute to its taste. Cultured monolayers, even if compacted together, cannot mimic the texture of traditional meat. Hence, contemporary products could be significantly limited by the disconnect between traditional and cultured meat taste and texture, making the endeavor of cultured meats potentially detrimental to companies. Therefore, there exists a need for the production of a thicker lab-cultured “clean” meat product with improved taste and texture.

### Electrospinning Fibers

Electrospinning is a method which may be used to process a polymer solution into a fiber. In embodiments wherein the diameter of the resulting fiber is on the nanometer scale, the fiber may be referred to as a nanofiber. Fibers may be formed into a variety of shapes by using a range of receiving surfaces, such as mandrels or collectors. In some embodiments, a flat shape, such as a sheet or sheet-like fiber mold, a fiber scaffold and/or tube, or a tubular lattice, may be formed by using a substantially round or cylindrical mandrel. In certain embodiments, the electrospun fibers may be cut and/or unrolled from the mandrel as a fiber mold to form the sheet. The resulting fiber molds or shapes may be used in many applications, including filters and the like.

Electrospinning methods may involve spinning a fiber from a polymer solution by applying a high DC voltage potential between a polymer injection system and a mandrel. In some embodiments, one or more charges may be applied to one or more components of an electrospinning system. In some embodiments, a charge may be applied to the mandrel, the polymer injection system, or combinations or portions thereof. Without wishing to be bound by theory, as the polymer solution is ejected from the polymer injection system, it is thought to be destabilized due to its exposure to a charge. The destabilized solution may then be attracted to a charged mandrel. As the destabilized solution moves from the polymer injection system to the mandrel, its solvents may evaporate and the polymer may stretch, leaving a long, thin fiber that is deposited onto the mandrel. The polymer solution may form a Taylor cone as it is ejected from the polymer injection system and exposed to a charge.

In certain embodiments, a first polymer solution comprising a first polymer and a second polymer solution comprising a second polymer may each be used in a separate polymer injection system at substantially the same time to produce one or more electrospun fibers comprising the first polymer interspersed with one or more electrospun fibers comprising the second polymer. Such a process may be referred to as “co-spinning” or “co-electrospinning,” and a scaffold produced by such a process may be described as a co-spun or co-electrospun scaffold.

### Polymer Injection System

A polymer injection system may include any system configured to eject some amount of a polymer solution into an atmosphere to permit the flow of the polymer solution from the injection system to the mandrel. In some embodiments, the polymer injection system may deliver a continuous or linear stream with a controlled volumetric flow rate of a polymer solution to be formed into a fiber. In some embodiments, the polymer injection system may deliver a variable stream of a polymer solution to be formed into a fiber. In some embodiments, the polymer injection system may be configured to deliver intermittent streams of a polymer solution to be formed into multiple fibers. In some embodiments, the polymer injection system may include a syringe under manual or automated control. In some embodiments, the polymer injection system may include multiple syringes and multiple needles or needle-like components under individual or combined manual or automated control. In some embodiments, a multi-syringe polymer injection system may include multiple syringes and multiple needles or needle-like components, with each syringe containing the same polymer solution. In some embodiments, a multi-syringe polymer injection system may include multiple syringes and multiple needles or needle-like components, with each syringe containing a different polymer solution. In some embodiments, a charge may be applied to the polymer injection system, or to a portion thereof. In some embodiments, a charge may be applied to a needle or needle-like component of the polymer injection system.

In some embodiments, the polymer solution may be ejected from the polymer injection system at a flow rate of less than or equal to about 5 mL/h per needle. In other embodiments, the polymer solution may be ejected from the polymer injection system at a flow rate per needle in a range from about 0.01 mL/h to about 50 mL/h. The flow rate at which the polymer solution is ejected from the polymer injection system per needle may be, in some non-limiting examples, about 0.01 mL/h, about 0.05 mL/h, about 0.1 mL/h, about 0.5 mL/h, about 1 mL/h, about 2 mL/h, about 3 mL/h, about 4 mL/h, about 5 mL/h, about 6 mL/h, about 7 mL/h, about 8 mL/h, about 9 mL/h, about 10 mL/h, about 11 mL/h, about 12 mL/h, about 13 mL/h, about 14 mL/h, about 15 mL/h, about 16 mL/h, about 17 mL/h, about 18 mL/h, about 19 mL/h, about 20 mL/h, about 21 mL/h, about 22 mL/h, about 23 mL/h, about 24 mL/h, about 25 mL/h, about 26 mL/h, about 27 mL/h, about 28 mL/h, about 29 mL/h, about 30 mL/h, about 31 mL/h, about 32 mL/h, about 33 mL/h, about 34 mL/h, about 35 mL/h, about 36 mL/h, about 37 mL/h, about 38 mL/h, about 39 mL/h, about 40 mL/h, about 41 mL/h, about 42 mL/h, about 43 mL/h, about 44 mL/h, about 45 mL/h, about 46 mL/h, about 47 mL/h, about 48 mL/h, about 49 mL/h, about 50 mL/h, or any range between any two of these values, including endpoints.

As the polymer solution travels from the polymer injection system toward the mandrel, the diameter of the resulting fibers may be in the range of about 100 nm to about 1500 nm. Some non-limiting examples of electrospun fiber diameters may include about 100 nm, about 150 nm, about 200 nm, about 250 nm, about 300 nm, about 350 nm, about 400 nm, about 450 nm, about 500 nm, about 550 nm, about 600 nm, about 650 nm, about 700 nm, about 750 nm, about 800 nm, about 850 nm, about 900 nm, about 950 nm, about 1,000 nm, about 1,050 nm, about 1,100 nm, about 1,150 nm, about 1,200 nm, about 1,250 nm, about 1,300 nm, about 1,350 nm, about 1,400 nm, about 1,450 nm, about 1,500 nm, or any range between any two of these values, including endpoints. In some embodiments, the electrospun fiber diameter may be from about 300 nm to about 1300 nm.

### Polymer Solution

In some embodiments, the polymer injection system may be filled with a polymer solution. In some embodiments, the polymer solution may comprise one or more polymers. In some embodiments, the polymer solution may be a fluid formed into a polymer liquid by the application of heat. A polymer solution may include, for example, non-resorbable polymers, resorbable polymers, natural polymers, or a combination thereof.

In some embodiments, the polymers may include, for example, nylon, nylon 6,6, polycaprolactone, polyethylene oxide terephthalate, polybutylene terephthalate, polyethylene oxide terephthalate-co-polybutylene terephthalate, polyethylene terephthalate, polyurethane, polyvinylpyrrolidone, polymethylmethacrylate, polyethylene, polyethylene oxide, polyacrylonitrile, silicone, polycarbonate, polylactide, polyglycolide, polyether ketone ketone, polyether ether ketone, polyether imide, polyamide, polystyrene, polyether sulfone, polysulfone, polyvinyl acetate, polytetrafluoroethylene, polyvinylidene fluoride, polylactic acid, polyglycolic acid, polylactide-co-glycolide, poly(lactide-co-caprolactone), polyglycerol sebacate, polydioxanone, polyhydroxybutyrate, poly-4-hydroxybutyrate, trimethylene carbonate, polydiols, polyesters, collagen, gelatin, fibrin, fibronectin, albumin, hyaluronic acid, elastin, chitosan, alginate, silk, zein, a soy protein, a plant protein, a carbohydrate, copolymers thereof, and combinations thereof. In some embodiments, the resulting electrospun polymer fiber may include a combination of one or more of a plant protein, a carbohydrate, and a synthetic polymer.

It may be understood that polymer solutions may also include a combination of one or more of non-resorbable, resorbable polymers, and naturally occurring polymers in any combination or compositional ratio. In an alternative embodiment, the polymer solutions may include a combination of two or more non-resorbable polymers, two or more resorbable polymers or two or more naturally occurring polymers. In some non-limiting examples, the polymer solution may comprise a weight percent ratio of, for example, from about 5% to about 90%. Non-limiting examples of such weight percent ratios may include about 5%, about 10%, about 15%, about 20%, about 25%, about 30%, about 33%, about 35%, about 40%, about 45%, about 50%, about 55%, about 60%, about 66%, about 70%, about 75%, about 80%, about 85%, about 90%, or ranges between any two of these values, including endpoints.

In some embodiments, the polymer solution may comprise one or more solvents. In some embodiments, the solvent may comprise, for example, polyvinylpyrrolidone, hexafluoro-2-propanol (HFIP), acetone, dimethylformamide, dimethylsulfoxide, N-methylpyrrolidone, N,N-dimethylformamide, Nacetonitrile, hexanes, ether, dioxane, ethyl acetate, pyridine, toluene, xylene, tetrahydrofuran, trifluoroacetic acid, hexafluoroisopropanol, acetic acid, dimethylacetamide, chloroform, dichloromethane, water, alcohols, ionic compounds, or combinations thereof. The concentration range of polymer or polymers in solvent or solvents may be, without limitation, from about 1 wt % to about 50 wt %. Some non-limiting examples of polymer concentration in solution may include about 1 wt %, 3 wt %, 5 wt %, about 10 wt %, about 15 wt %, about 20 wt %, about 25 wt %, about 30 wt %, about 35 wt %, about 40 wt %, about 45 wt %, about 50 wt %, or ranges between any two of these values, including endpoints.

In some embodiments, the polymer solution may also include additional materials. Non-limiting examples of such additional materials may include fluorescent materials, luminescent materials, antibiotics, growth factors, vitamins, cytokines, steroids, anti-inflammatory drugs, small molecules, sugars, salts, peptides, proteins, cell factors, DNA, RNA, fats, proteins, carbohydrates, minerals, or any combination thereof. In some embodiments, the additional material may have nutritional value.

In some embodiments, the additional materials may be present in the polymer solution or in the resulting electrospun polymer fibers in an amount from about 1 wt % to about 1500 wt % of the polymer mass. In some non-limiting examples, the additional materials may be present in the polymer solution or in the resulting electrospun polymer fibers in an amount of about 1 wt %, about 5 wt %, about 10 wt %, about 15 wt %, about 20 wt %, about 25 wt %, about 30 wt %, about 35 wt %, about 40 wt %, about 45 wt %, about 50 wt %, about 55 wt %, about 60 wt %, about 65 wt %, about 70 wt %, about 75 wt %, about 80 wt %, about 85 wt %, about 90 wt %, about 95 wt %, about 100 wt %, about 125 wt %, about 150 wt %, about 175 wt %, about 200 wt %, about 225 wt %, about 250 wt %, about 275 wt %, about 300 wt %, about 325 wt %, about 350 wt %, about 375 wt %, about 400 wt %, about 425 wt %, about 450 wt %, about 475 wt %, about 500 wt %, about 525 wt %, about 550 wt %, about 575 wt %, about 600 wt %, about 625 wt %, about 650 wt %, about 675 wt %, about 700 wt %, about 725 wt %, about 750 wt %, about 775 wt %, about 800 wt %, about 825 wt %, about 850 wt %, about 875 wt %, about 900 wt %, about 925 wt %, about 950 wt %, about 975 wt %, about 1000 wt %, about 1025 wt %, about 1050 wt %, about 1075 wt %, about 1100 wt %, about 1125 wt %, about 1150 wt %, about 1175 wt %, about 1200 wt %, about 1225 wt %, about 1250 wt %, about 1275 wt %, about 1300 wt %, about 1325 wt %, about 1350 wt %, about 1375 wt %, about 1400 wt %, about 1425 wt %, about 1450 wt %, about 1475 wt %, about 1500 wt %, or any range between any of these two values, including endpoints.

### Applying Charges to Electrospinning Components

In an electrospinning system, one or more charges may be applied to one or more components, or portions of components, such as, for example, a mandrel or a polymer injection system, or portions thereof. In some embodiments, a positive charge may be applied to the polymer injection system, or portions thereof. In some embodiments, a negative charge may be applied to the polymer injection system, or portions thereof. In some embodiments, the polymer injection system, or portions thereof, may be grounded. In some embodiments, a positive charge may be applied to mandrel, or portions thereof. In some embodiments, a negative charge may be applied to the mandrel, or portions thereof. In some embodiments, the mandrel, or portions thereof, may be grounded. In some embodiments, one or more components or portions thereof may receive the same charge. In some embodiments, one or more components, or portions thereof, may receive one or more different charges.

The charge applied to any component of the electrospinning system, or portions thereof, may be from about-15 kV to about 30 kV, including endpoints. In some non-limiting examples, the charge applied to any component of the electrospinning system, or portions thereof, may be about-15 kV, about-10 kV, about-5 kV, about-4 kV, about-3 kV, about-1 kV, about-0.01 kV, about 0.01 kV, about 1 kV, about 5 kV, about 10 kV, about 11 kV, about 11.1 kV, about 12 kV, about 15 kV, about 20 kV, about 25 kV, about 30 kV, or any range between any two of these values, including endpoints. In some embodiments, any component of the electrospinning system, or portions thereof, may be grounded.

### Mandrel Movement During Electrospinning

During electrospinning, in some embodiments, the mandrel may move with respect to the polymer injection system. In some embodiments, the polymer injection system may move with respect to the mandrel. The movement of one electrospinning component with respect to another electrospinning component may be, for example, substantially rotational, substantially translational, or any combination thereof. In some embodiments, one or more components of the electrospinning system may move under manual control. In some embodiments, one or more components of the electrospinning system may move under automated control. In some embodiments, the mandrel may be in contact with or mounted upon a support structure that may be moved using one or more motors or motion control systems. The pattern of the electrospun fiber deposited on the mandrel may depend upon the one or more motions of the mandrel with respect to the polymer injection system. In some embodiments, the mandrel surface may be configured to rotate about its long axis. In one non-limiting example, a mandrel having a rotation rate about its long axis that is faster than a translation rate along a linear axis, may result in a nearly helical deposition of an electrospun fiber, forming windings about the mandrel. In another example, a mandrel having a translation rate along a linear axis that is faster than a rotation rate about a rotational axis, may result in a roughly linear deposition of an electrospun fiber along a liner extent of the mandrel.

### Electrospun Polymer Fibers for Cultured Meat Production

Scaffolds of various sizes and thicknesses may help solve the engineering problems that cultured meat products currently face. In general, using a cellular engineering process that involves cells and such a scaffold may allow for the migration of the cells throughout the entirety of the scaffold. However, many existing scaffolds fail to provide the correct representation of the extracellular matrix.

Electrospun polymer fibers may provide solutions to these challenges. Electrospun polymer fibers may be used to create scaffolds of various sizes and thicknesses. In contrast to scaffolds made from other materials, electrospun polymer fibers may be formed into a variety of shapes, including discs, tubes, sheets, and the like, making them easy to fit into existing cell culture devices. The use of electrospun polymer fiber scaffolds may allow the creation of a higher volume of cultured meat using existing equipment. Moreover, electrospun fiber scaffolds could be used to develop products with specific structures (including meats like steaks or sashimi, for example), targeting a specific volume and cellular environment for the final product. Electrospun polymer fibers can be used, for example, to create a scaffold having highly aligned fibers. Such aligned fibers may provide the necessary topographical and electrical cues to cells in culture, providing appropriate stimulation for the development of engineered musculoskeletal tissue.

Furthermore, and without wishing to be bound by theory, it is thought that some of the taste in traditional slaughtered meat is the result of lactate or lactic acid. Lactic acid is produced in two instances: in times of high stress, and during anaerobic respiration. Research has suggested that post-mortem, muscle cells continue to operate for a short period of time from anaerobic respiration. The lactic acid produced during that period is thought to drop the pH of the meat to around 5.5, although a wider range of pH values may be found in different meats. Electrospun polymer fibers can be engineered to specifically deteriorate or dissolve over a period of time into chemical byproducts naturally found in the body, including lactic acid, glycolic acid, and caproic acid. The period of time can range depending on the planned end product, and can be anywhere from about 1 day to about 6 weeks. The dissolution of electrospun polymer fibers into these chemical byproducts may create a more acidic environment that would lead to an improved cultured meat product. A small drop in the pH of the cell environment may also encourage healthy, organized tissue growth. Accordingly, a decrease in pH during culturing could lead to improved tissue growth (and thereby improved texture), as well as improved taste of the cultured meat product.

Furthermore, electrospun polymer fibers may be made from various different polymers, as described above, and these different polymers may be used to promote different cell differentiation and/or proliferation properties for different components of cultured meat, including myocytes, adipocytes, and chondrocytes in muscle, fat, and connective tissue, respectively. These different tissue types differentiate stem cells in their own unique ways based on different environmental and/or chemical signals. Electrospun polymer fibers could be used to create a scaffold having different sections with different properties, each section designed to generate and support a desired tissue type. Electrospun polymer fibers can be manufactured with different moduli, diameters, surface textures, surface chemical interactions, or spatially controlled drug delivery systems. In short, electrospun polymer fibers could be used to create cultured meat products that look, feel, and taste like traditional slaughtered meats.

In some embodiments, the cultured meat products described herein may comprise a scaffold and a population of cells. The population of cells may include, in some non-limiting examples, mesenchymal stem cells, myocytes, adipocytes, chondrocytes, osteoblasts, or any combination thereof. Publications that demonstrate the culture of myocytes, adipocytes chondrocytes, and osteoblasts on electrospun polymer fibers include: (1) Khan et al. Evaluation of Changes in Morphology and Function of Human Induced Pluripotent Stem Cell Derived Cardiomyocytes (HiPSC-CMs) Cultured on an Aligned-Nanofiber Cardiac Patch. PLOS One. 2015; 10(5):e0126338. doi:10.1371/journal/pone.0126338, which is incorporated herein by reference; and (2) Pandey et al. Aligned Nanofiber Material Supports Cell Growth and Increases Osteogenesis in Canine Adipose-Derived Mesenchymal Stem Cells In Vitro. J Biomed Mater Res Part A 2018, 106A:1780-1788, which is incorporated herein by reference.

The scaffold may comprise an electrospun polymer fiber as described herein. In some embodiments, the electrospun polymer fiber may comprise a polymer selected from nylon, nylon 6,6, polycaprolactone, polyethylene oxide terephthalate, polybutylene terephthalate, polyethylene oxide terephthalate-co-polybutylene terephthalate, polyethylene terephthalate, polyurethane, polyethylene, polyethylene oxide, polyvinylpyrrolidone, polymethylmethacrylate, polyacrylonitrile, silicone, polycarbonate, polylactide, polyglycolide, polyether ketone ketone, polyether ether ketone, polyether imide, polyamide, polystyrene, polyether sulfone, polysulfone, polyvinyl acetate, polytetrafluoroethylene, polyvinylidene fluoride, polylactic acid, polyglycolic acid, polylactide-co-glycolide, poly(lactide-co-caprolactone), polyglycerol sebacate, polydioxanone, polyhydroxybutyrate, poly-4-hydroxybutyrate, trimethylene carbonate, polydiols, polyesters, collagen, gelatin, fibrin, fibronectin, albumin, hyaluronic acid, elastin, chitosan, alginate, silk, zein, a soy protein, a plant protein, a carbohydrate, copolymers thereof, and combinations thereof. In some embodiments, the resulting electrospun polymer fiber may include a combination of one or more of a plant protein, a carbohydrate, and a synthetic polymer.

In certain embodiments, the electrospun polymer fiber may comprise multiple electrospun polymer fibers aligned substantially parallel to one another, as described herein. In other embodiments, the electrospun fiber may comprise multiple electrospun polymer fibers having different orientations relative to one another, including randomly oriented, substantially parallel, and combinations thereof, as described herein. In embodiments having multiple electrospun polymer fibers, the multiple electrospun polymer fibers may have multiple orientations and/or multiple fiber diameters, as described herein, and may comprise one or more polymers, as described herein. In certain embodiments, a scaffold may comprise multiple co-spun electrospun polymer fibers, as described herein.

In some embodiments, the scaffold may further comprise one or more electrospun polymer fiber fragments. The electrospun polymer fiber fragments may be, for example, dispersed throughout the scaffold, or dispersed throughout a particular portion of the scaffold. Without wishing to be bound by theory, the electrospun polymer fiber fragments may aid or support the culturing and expansion of cells within the scaffold. In some embodiments, the electrospun polymer fiber fragments may have a length from about 100 μm to about 10 mm. In certain embodiments, the electrospun polymer fiber fragments may have a maximum length of about 1 mm.

In certain embodiments, the scaffold may comprise one or more electrospun polymer fiber types, and the one or more electrospun polymer fiber types may be co-spun. In an embodiment, each electrospun fiber type may be suitable to support the differentiation of one or more cells into a different biological tissue. For example, a scaffold may comprise a first electrospun polymer fiber type suitable to support the differentiation of cells into muscle, a second electrospun polymer fiber type suitable to support the differentiation of cells into bone, a third electrospun polymer fiber type suitable to support the differentiation of cells into cartilage, a fourth electrospun polymer fiber type suitable to support the differentiation of cells into a connective tissue, a fifth electrospun polymer fiber type suitable to support the differentiation of cells into a blood vessel, or any combination of these electrospun polymer fiber types.

A scaffold may include, in one non-limiting example, a first plurality of electrospun polymer fibers comprising a polymer and having a diameter and/or orientation to support the proliferation of a first type of cells; a second plurality of electrospun polymer fibers comprising a polymer and having a diameter and/or orientation to support the proliferation of a second type of cells; a third plurality of electrospun polymer fibers comprising a polymer and having a diameter and/or orientation to support the proliferation of a third type of cells; a fourth plurality of electrospun polymer fibers comprising a polymer and having a diameter and/or orientation to support the proliferation of a fourth type of cells; and so on. In some embodiments, the first, second, third, and fourth types of cells in such embodiments may include any mammalian cells, such as muscle cells, vascular cells, fat cells, connective tissue cells, neural cells, or combinations thereof.

In some embodiments, the electrospun polymer fiber may comprise a polymer configured to degrade to produce a byproduct. In certain embodiments, the byproduct may include, for example, lactic acid, glycolic acid, caproic acid, and combinations thereof. In some embodiments, the electrospun polymer fiber may be configured to degrade upon exposure to a substance; in one non-limiting example, the substance may comprise saliva.

In certain embodiments, the electrospun polymer fiber may comprise an additional material, as described herein, and may be configured to release at least a portion of the additional material upon the application of a mechanical force. In one embodiment, the mechanical force may be produce by actions such as chewing, cutting, breaking, or combinations thereof. In some embodiments, the cultured meat product may include an intact electrospun polymer fiber, while in other embodiments, the electrospun polymer fiber of the scaffold may be completely or nearly completely resorbed in the final cultured meat product. In an embodiment, the intact electrospun polymer fiber may be configured to mimic the texture and/or other properties of traditional slaughtered meat.

In certain embodiments, the cultured meat product may have a thickness from about 100 μm to about 500 mm. The thickness may be, for example, about 100 μm, about 200 μm, about 300 μm, about 400 μm, about 500 μm, about 600 μm, about 700 μm, about 800 μm, about 900 μm, about 1 mm, about 5 mm, about 10 mm, about 25 mm, about 50 mm, about 75 mm, about 100 mm, about 125 mm, about 150 mm, about 175 mm, about 200 mm, about 225 mm, about 250 mm, about 275 mm, about 300 mm, about 325 mm, about 350 mm, about 375 mm, about 400 mm, about 425 mm, about 450 mm, about 475 mm, about 500 mm, or any range between any two of these values, including endpoints. In some embodiments, the cultured meat product may have a thickness from about 5 mm to about 75 mm. In an embodiment, the thickness may be about 25 mm.

In some embodiments, the cultured meat products described herein may be configured to mimic or closely resemble a property of a traditional slaughtered meat. The property may include, for example, taste, texture, size, shape, topography, or any combination thereof.

In some embodiments, a method of producing a cultured meat product may comprise preparing a scaffold as described herein, placing the scaffold into a bioreactor, adding a population of cells to the bioreactor, culturing the population of cells in the bioreactor containing the scaffold for a period of time, thereby forming the cultured meat product, and removing the cultured meat product from the bioreactor. In embodiments, the cultured meat product may have the characteristics and features of the cultured meat products described herein. In some embodiments, the scaffold and population of cells may each have the characteristics and features of the scaffolds and populations of cells described herein.

In some embodiments, the step of culturing the population of cells in the bioreactor may be carried out for a period of time. The period of time could be, for example, about 1 day, about 2 days, about 3 days, about 4 days, about 5 days, about 6 days, about 1 week, about 1.5 weeks, about 2 weeks, about 2.5 weeks, about 3 weeks, about 3.5 weeks, about 4 weeks, about 4.5 weeks, about 5 weeks, about 5.5 weeks, about 6 weeks, or any range between any two of these values, including endpoints. In one embodiment, the period of time may be about 3 weeks.

### EXAMPLES

### Example 1: Zein-Containing Scaffolds for Cultured Meat Products

Electrospun zein as a plant-based protein component of a scaffold was investigated for inclusion in a cultured meat product, as described herein. 90% ethanol in distilled water quickly dissolved zein powder. This 90% aqEtOH solution was able to produce zein fibers with electrospinning, but the electrospinning process was not sufficiently stable for zein-only fibers.

To improve the stability of the electrospinning process with zein, an additional polymer component was investigated for combination with the zein in solution. Two polymers were particularly attractive as candidates: polyethylene oxide (PEO; both 1M Mw and 100k Mw PEO polymer resins were tested) and a 50/50 DL-lactide/glycolide copolymer (PDLGA 5010). Both of these polymers are safe to consume, bioresorb quickly, and are fairly elastic.

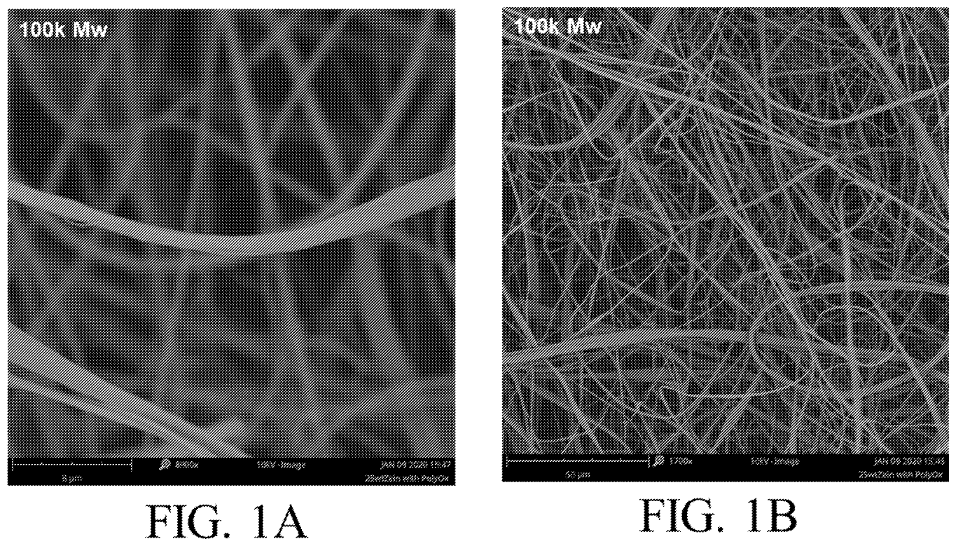

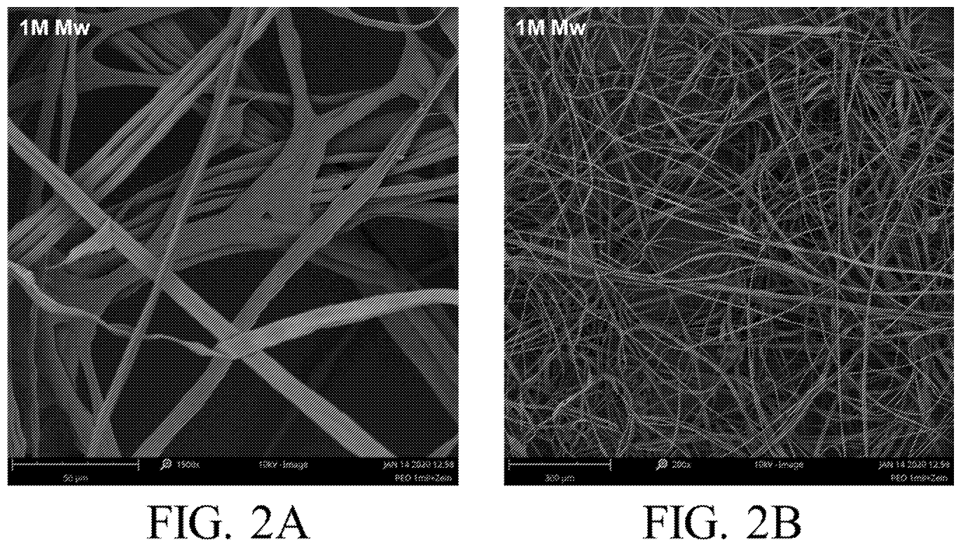

PEO+zein. Both of the PEO+zein solutions experienced significant improvements to the electrospinning process. Both molecular weights (Mw) of PEO formed fibers that were a majority zein by mass. The 100k Mw PEO+zein yielded more cylindrical fibers, while the 1M Mw PEO+zein yielded fiber bundles and ribbon-like fibers. Both scaffolds appeared to be fairly porous with some zein agglomerates dispersed in the scaffold. FIG. 1A shows an SEM image (8900×) of a scaffold electrospun using a 100k Mw PEO+zein solution, as described above, and FIG. 1B shows an SEM image (1700×) of the scaffold of FIG. 1A. FIG. 1A and FIG. 1B both show relatively cylindrical fibers, as described above. FIG. 2A shows an SEM image (1500×) of a scaffold electrospun using a 1M Mw PEO+zein solution, as described above, and FIG. 2B shows an SEM image (200×) of the scaffold of FIG. 2A. FIG. 2A and FIG. 2B both show ribbon-like fibers, as described above.

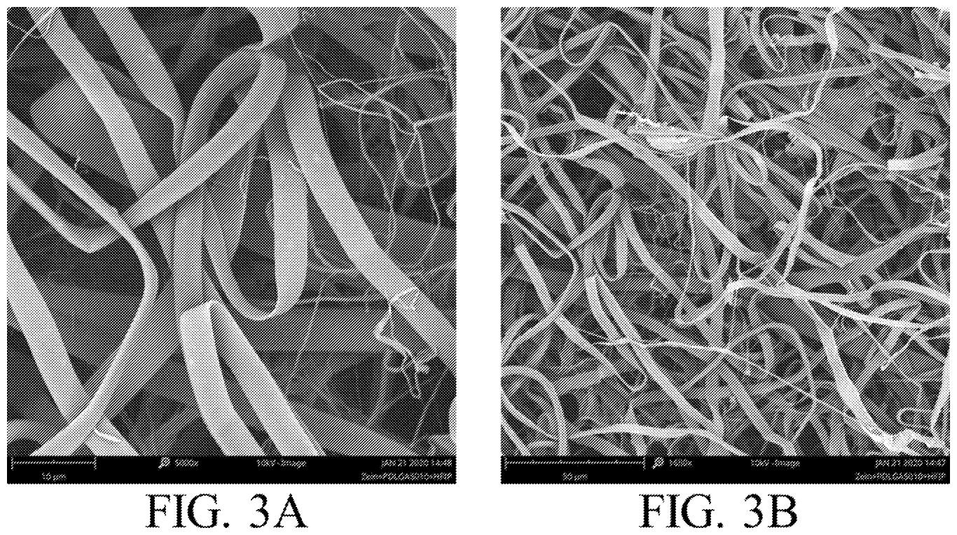

PDLGA 5010+zein. PDLGA 5010 was added to zein powder in HFIP to aid the production of a zein-containing scaffold. The solution was electrospun to form a scaffold. FIG. 3A shows an SEM image (5000×) of a scaffold electrospun using a PDLGA 5010+zein solution, as described above. FIG. 3B shows an SEM image (1650×) of the scaffold of FIG. 3A. FIG. 3A and FIG. 3B both show ribbon-like fibers.

Overall, and without wishing to be bound by theory, the addition of zein to electrospun polymer fibers, as described above, may accelerate the rate of cellular growth when a scaffold comprising such fibers is used to culture cells for meat products. In addition, if the cultured cells do not entirely consume the zein within the scaffold the zein is a plant-based protein that is safe for consumption.

### Example 2: Soy Protein Isolate-Containing Scaffolds for Cultured Meat Products

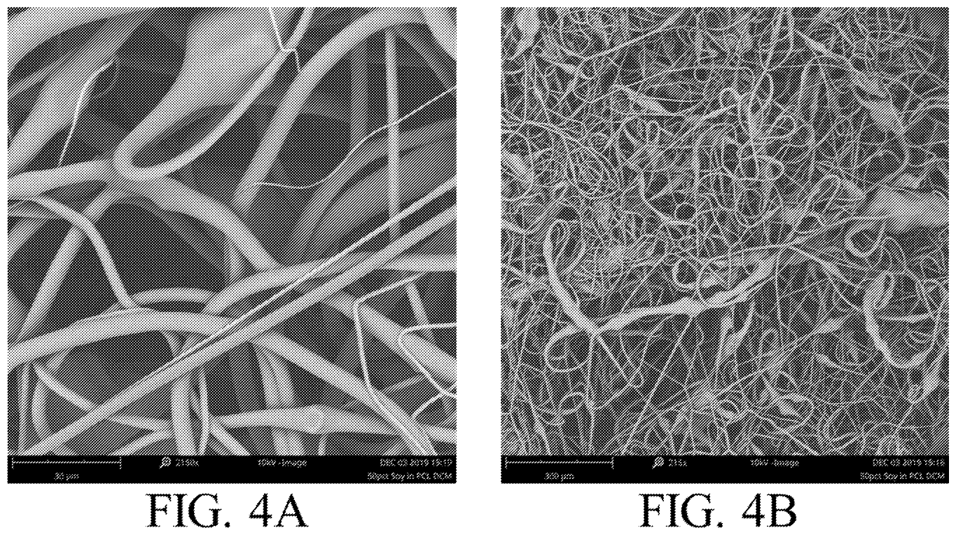

Soy protein isolate was added to a polycaprolactone (PCL) solution at 50% of the mass of the PCL to create electrospun polymer fibers having about 33% of the final dry mass from soy protein isolate and about 67% of the final dry mass from PCL. This combination produced a sheet of material with substantial mechanical integrity. The resulting average fiber diameter was about 6.5 μm, and the soy protein isolate appeared to be a significant part of the fibers. While large agglomerates of the soy protein isolates appeared, they appeared to be incorporated into a fiber or a fiber-like structure. The resulting fibers also appeared to maintain a fair degree of porosity. FIG. 4A shows an SEM image (2150×) of a scaffold electrospun using a PCL+soy protein isolate solution, as described above. FIG. 4B shows an SEM image (215×) of the scaffold of FIG. 4A.

While the present disclosure has been illustrated by the description of exemplary embodiments thereof, and while the embodiments have been described in certain detail, it is not the intention of the Applicant to restrict or in any way limit the scope of the appended claims to such detail. Additional advantages and modifications will readily appear to those skilled in the art. Therefore, the disclosure in its broader aspects is not limited to any of the specific details, representative devices and methods, and/or illustrative examples shown and described. Accordingly, departures may be made from such details without departing from the spirit or scope of the Applicant's general inventive concept.

1. A cultured meat product comprising:
a scaffold comprising an electrospun polymer fiber; and
a population of cells;
wherein the cultured meat product has a thickness from about 100 μm to about 500 mm.

2. The cultured meat product of claim 1, wherein the electrospun polymer fiber comprises a polymer selected from the group consisting of nylon, nylon 6,6, polycaprolactone, polyethylene oxide terephthalate, polybutylene terephthalate, polyethylene oxide terephthalate-co-polybutylene terephthalate, polyethylene terephthalate, polyurethane, polyethylene, polyethylene oxide, polyvinylpyrrolidone, polymethylmethacrylate, polyacrylonitrile, silicone, polycarbonate, polylactide, polyglycolide, polyether ketone ketone, polyether ether ketone, polyether imide, polyamide, polystyrene, polyether sulfone, polysulfone, polyvinyl acetate, polytetrafluoroethylene, polyvinylidene fluoride, polylactic acid, polyglycolic acid, polylactide-co-glycolide, poly(lactide-co-caprolactone), polyglycerol sebacate, polydioxanone, polyhydroxybutyrate, poly-4-hydroxybutyrate, trimethylene carbonate, polydiols, polyesters, collagen, gelatin, fibrin, fibronectin, albumin, hyaluronic acid, elastin, chitosan, alginate, silk, zein, a soy protein, a plant protein, a carbohydrate, copolymers thereof, and combinations thereof.

3. The cultured meat product of claim 1, wherein the electrospun polymer fiber comprises a polymer configured to degrade to produce a byproduct.

4. The cultured meat product of claim 3, wherein the byproduct is selected from the group consisting of lactic acid, glycolic acid, caproic acid, and combinations thereof.

5. The cultured meat product of claim 1, wherein the electrospun polymer fiber comprises a polymer configured to degrade upon exposure to saliva.

6. The cultured meat product of claim 1, wherein the scaffold comprises multiple electrospun polymer fibers aligned substantially parallel to one another.

7. The cultured meat product of claim 1, wherein the thickness of the cultured meat product is from about 5 mm to about 75 mm.

8. The cultured meat product of claim 1, wherein the cultured meat product is configured to mimic a property of a traditional slaughtered meat.

9. The cultured meat product of claim 8, wherein the property is selected from the group consisting of taste, texture, size, shape, topography, and combinations thereof.

10. The cultured meat product of claim 1, wherein the population of cells is selected from the group consisting of mesenchymal stem cells, myocytes, adipocytes, chondrocytes, and combinations thereof.

11. The cultured meat product of claim 1, wherein the scaffold further comprises a plurality of electrospun fiber fragments having a maximum length of about 10 mm.

12. A method of producing a cultured meat product, the method comprising:
preparing a scaffold comprising an electrospun polymer fiber;
placing the scaffold into a bioreactor;
adding a population of cells to the bioreactor;
culturing the population of cells in the bioreactor containing the scaffold for a period of time, thereby forming the cultured meat product having a thickness from about 100 μm to about 500 mm; and
removing the cultured meat product from the bioreactor.

13. The method of claim 12, wherein the period of time is from about 1 day to about 6 weeks.

14. The method of claim 12, wherein the period of time is about 3 weeks.

15. The method of claim 12, wherein the electrospun polymer fiber comprises a polymer selected from the group consisting of nylon, nylon 6,6, polycaprolactone, polyethylene oxide terephthalate, polybutylene terephthalate, polyethylene oxide terephthalate-co-polybutylene terephthalate, polyethylene terephthalate, polyurethane, polyethylene, polyethylene oxide, polyvinylpyrrolidone, polymethylmethacrylate, polyacrylonitrile, silicone, polycarbonate, polylactide, polyglycolide, polyether ketone ketone, polyether ether ketone, polyether imide, polyamide, polystyrene, polyether sulfone, polysulfone, polyvinyl acetate, polytetrafluoroethylene, polyvinylidene fluoride, polylactic acid, polyglycolic acid, polylactide-co-glycolide, poly(lactide-co-caprolactone), polyglycerol sebacate, polydioxanone, polyhydroxybutyrate, poly-4-hydroxybutyrate, trimethylene carbonate, polydiols, polyesters, collagen, gelatin, fibrin, fibronectin, albumin, hyaluronic acid, elastin, chitosan, alginate, silk, zein, a soy protein, a plant protein, a carbohydrate, copolymers thereof, and combinations thereof.

16. The method of claim 12, wherein the electrospun polymer fiber comprises a polymer configured to degrade to produce a byproduct.

17. The method of claim 16, wherein the byproduct is selected from the group consisting of lactic acid, glycolic acid, caproic acid, and combinations thereof.

18. The method of claim 12, further comprising exposing the electrospun polymer fiber to saliva, wherein the electrospun polymer fiber comprises a polymer configured to degrade upon exposure to saliva.

19. The method of claim 12, wherein the scaffold comprises multiple electrospun polymer fibers aligned substantially parallel to one another.

20. The method of claim 12, wherein the thickness of the cultured meat product is from about 5 mm to about 75 mm.

21. The method of claim 12, wherein the cultured meat product is configured to mimic a property of a traditional slaughtered meat.

22. The method of claim 21, wherein the property is selected from the group consisting of taste, texture, size, shape, topography, and combinations thereof.

23. The method of claim 12, wherein the population of cells is selected from the group consisting of mesenchymal stem cells, myocytes, adipocytes, chondrocytes, and combinations thereof.

24. The method of claim 12, wherein the scaffold further comprises a plurality of electrospun fiber fragments having a maximum length of about 10 mm.
